(19) United States
(12) Patent Application Publication
(10) Pub. No.: US 2023/0271967 A1
(43) Pub. Date: Aug. 31, 2023

(54) SOLID STATE FORMS OF FEZOLINETANT AND SALTS THEREOF

(71) Applicant: TEVA PHARMACEUTICALS INTERNATIONAL GMBH, Jona (CH)

(72) Inventors: Helena Ceric, Zagreb (HR); Nikolina Janton, Jakovlje (HR)

(21) Appl. No.: 18/015,736

(22) PCT Filed: Aug. 4, 2021
(86) PCT No.: PCT/US2021/044436

§ 371 Date: Jan. 12, 2023

Related U.S. Application Data
(60) Provisional application No. 63/060,714, filed on Aug. 4, 2020.
(60) Provisional application No. 63/070,335, filed on Aug. 26, 2020.
(60) Provisional application No. 63/093,844, filed on Oct. 20, 2020.
(60) Provisional application No. 63/114,021, filed on Nov. 16, 2020.
(60) Provisional application No. 63/130,443, filed on Dec. 24, 2020.

(51) Int. Cl. C07D 487/04

(52) U.S. Cl. CPC C07D 487/04; C07B 2200/13

(57) ABSTRACT

The present disclosure encompasses solid state forms of Fezolinetant, including salts and cocrystals of Fezolinetant, in embodiments crystalline polymorphs of Fezolinetant, processes for preparation thereof, and pharmaceutical compositions thereof.

### FIELD OF THE DISCLOSURE

The present disclosure encompasses solid state forms of Fezolinetant, including salts and cocrystals of Fezolinetant, in embodiments crystalline polymorphs of Fezolinetant, processes for preparation thereof, and pharmaceutical compositions thereof.

### BACKGROUND OF THE DISCLOSURE

Fezolinetant, (4-fluorophenyl)-[(8R)-8-methyl-3-(3-methyl-1,2,4-thiadiazol-5-yl)-6,8-dihydro-5H-[1,2,4]triazolo[4,3-a]pyrazin-7-yl]methanone, has the following chemical structure:

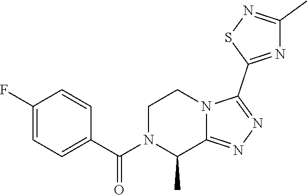 [SMILES: Cc1nsc(-c2nnc3n2CCN(C(=O)c2ccc(F)cc2)[C@@H]3C)n1]

Fezolinetant, also known as ESN364, is reported to be an antagonist of the GPCR known as the tachykinin NK3 receptor, and it is developed for the treatment of menopausal hot flashes (HF) and/or other menopausal symptoms such as night sweats and/or sleep and mood disturbances. Fezolinetant has recently been investigated for the treatment of polycystic ovary syndrome (PCOS), endometriosis, benign prostate hyperplasia, and uterine fibroids.

The compound and processes for preparation thereof are described in International Publication No. WO 2014/154895, International Publication No. WO 2016/046398 and ACS Med. Chem. Lett. 2015, 6, 736-740.

Salts and crystals of the salts are described in International Publication No. WO 2019/074081.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Fezolinetant.

### SUMMARY OF THE DISCLOSURE

The present disclosure provides crystalline polymorphs and amorphous form of Fezolinetant, solid state forms of Fezolinetant salts, co-crystal forms of Fezolinetant, processes for preparation thereof, and pharmaceutical compositions thereof. These crystalline polymorphs and amorphous form can be used to prepare other solid state forms of Fezolinetant, Fezolinetant salts and their solid state forms.

The present disclosure also provides uses of the said solid state forms of Fezolinetant, solid state forms of Fezolinetant salts and co-crystal forms of Fezolinetant, in the preparation of other solid state forms of Fezolinetant, cocrystals or salts thereof.

The present disclosure provides crystalline polymorphs and amorphous form of Fezolinetant, solid state forms of Fezolinetant salts and co-crystal forms of Fezolinetant, for use in medicine, including for the treatment of menopausal hot flashes (HF) and/or other menopausal symptoms such as night sweats, and/or sleep and mood disturbances; polycystic ovary syndrome (PCOS); endometriosis; benign prostate hyperplasia; and uterine fibroids.

The present disclosure also encompasses the use of crystalline polymorphs or amorphous form of Fezolinetant, solid state forms of Fezolinetant salts and co-crystal forms of Fezolinetant of the present disclosure for the preparation of pharmaceutical compositions and/or formulations.

In another aspect, the present disclosure provides pharmaceutical compositions comprising crystalline polymorphs and/or amorphous form of Fezolinetant, solid state forms of Fezolinetant salts and co-crystal forms of Fezolinetant according to the present disclosure.

The present disclosure includes processes for preparing the above mentioned pharmaceutical compositions. The processes include combining any one or a combination of the crystalline polymorphs and amorphous form of Fezolinetant, solid state forms of Fezolinetant salts and co-crystal forms of Fezolinetant with at least one pharmaceutically acceptable excipient.

The crystalline polymorph and amorphous form of Fezolinetant, solid state forms of Fezolinetant salts and co-crystal forms of Fezolinetant as defined herein and the pharmaceutical compositions or formulations of the crystalline polymorph of Fezolinetant, solid state forms of Fezolinetant salts and co-crystal forms of Fezolinetant may be used as medicaments, such as for the treatment of menopausal hot flashes (HF), and/or other menopausal symptoms such as night sweats, and/or sleep and mood disturbances; polycystic ovary syndrome (PCOS); endometriosis; benign prostate hyperplasia; and uterine fibroids.

The present disclosure also provides methods of treating menopausal hot flashes (HF), and/or other menopausal symptoms such as night sweats, and/or sleep and mood disturbances; polycystic ovary syndrome (PCOS); endometriosis; benign prostate hyperplasia; or uterine fibroids, by administering a therapeutically effective amount of any one or a combination of the crystalline polymorphs and amorphous form of Fezolinetant, solid state forms of Fezolinetant salts, co-crystal forms of Fezolinetant of the present disclosure, or at least one of the above pharmaceutical compositions, to a subject suffering from menopausal hot flashes, and/or other menopausal symptoms such as night sweats, and/or sleep and mood disturbances; polycystic ovary syndrome (PCOS); endometriosis; benign prostate hyperplasia; and uterine fibroids, or otherwise in need of the treatment.

The present disclosure also provides uses of crystalline polymorphs or amorphous form of Fezolinetant of the present disclosure, solid state forms of Fezolinetant salts, co-crystal forms of Fezolinetant or at least one of the above pharmaceutical compositions, for the manufacture of medicaments for treating menopausal hot flashes (HF), and/or other menopausal symptoms such as night sweats, and/or sleep and mood disturbances; polycystic ovary syndrome (PCOS); endometriosis; benign prostate hyperplasia; and uterine fibroids.

### BRIEF DESCRIPTION OF THE DRAWINGS

FIG. 1 shows a characteristic X-ray powder diffraction pattern (XRPD) of Fezolinetant Form 2.

FIG. 2 shows a characteristic X-ray powder diffraction pattern (XRPD) of Fezolinetant Form 3.

FIG. 3 shows a characteristic X-ray powder diffraction pattern (XRPD) of amorphous Fezolinetant.

FIG. 4 shows a characteristic XRPD of Crystalline Form B of Fezolinetant:xinafoic acid.

FIG. 5 shows a characteristic XRPD of Crystalline Form C of Fezolinetant:xinafoic acid.

FIG. 6 shows a characteristic X-ray powder diffraction pattern (XRPD) of Fezolinetant esylate Form A.

FIG. 7 shows a characteristic X-ray powder diffraction pattern (XRPD) of Fezolinetant camsylate Form A.

FIG. 8 shows a characteristic X-ray powder diffraction pattern (XRPD) of Fezolinetant besylate Form B.

FIG. 9 shows a characteristic X-ray powder diffraction pattern (XRPD) of amorphous Fezolinetant tosylate.

FIG. 10 shows a characteristic X-ray powder diffraction pattern (XRPD) of Fezolinetant Form 4.

FIG. 11 shows a characteristic XRPD of Crystalline Form B of Fezolinetant: xinafoic acid.

FIG. 12 shows a characteristic XRPD of Crystalline Form D of Fezolinetant: xinafoic acid.

FIG. 13 shows a characteristic X-ray powder diffraction pattern (XRPD) of Fezolinetant Form 5.

FIG. 14 shows a characteristic FTIR spectrum for Fezolinetant Form 2.

FIG. 15 shows a characteristic solid state ¹³C NMR spectrum for Fezolinetant Form 2.

FIG. 16 shows a characteristic solid state ¹³C NMR spectrum for Fezolinetant:xinafoic acid Form C.

FIG. 17 shows a characteristic FTIR spectrum for Fezolinetant:xinafoic acid Form D.

FIG. 18 shows a characteristic solid state ¹³C NMR spectrum for Fezolinetant:xinafoic acid Form D.

FIG. 19 shows a characteristic FTIR spectrum for Fezolinetant Form 3.

FIG. 20 shows a characteristic FTIR spectrum for Fezolinetant:xinafoic acid Form C.

FIG. 21 shows a characteristic FTIR spectrum for Fezolinetant camsylate.

FIG. 22 shows a characteristic FTIR spectrum for Fezolinetant esylate.

### DETAILED DESCRIPTION OF THE DISCLOSURE

The present disclosure encompasses solid state forms of Fezolinetant, including crystalline polymorphs of Fezolinetant, solid state forms of Fezolinetant salts, co-crystal forms of Fezolinetant, processes for preparation thereof, and pharmaceutical compositions thereof.

Solid state properties of Fezolinetant and crystalline polymorphs thereof can be influenced by controlling the conditions under which Fezolinetant and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by)(RFD. Thus, a crystalline polymorph of Fezolinetant described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Fezolinetant. In some embodiments of the disclosure, the described crystalline polymorph of Fezolinetant may contain from about 1% to about 20% (w/w), from about 5% to about 20% (w/w), or from about 5% to about 10% (w/w) of one or more other crystalline polymorph of the same Fezolinetant.

Depending on which other crystalline polymorphs a comparison is made, the crystalline polymorphs of Fezolinetant of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Fezolinetant referred to herein as being characterized by graphical data “as depicted in” or “as substantially depicted in” a Figure will thus be understood to include any crystal forms of Fezolinetant characterized with the graphical data having such small variations, as are well known to the skilled person, in comparison with the Figure.

As used herein, the term “crystalline form of Fezolinetant” relates to a crystalline form of Fezolinetant base, salt or co-crystal.

“Co-Crystal” or “Cocrystal” as used herein is defined as a crystalline material including two or more molecules in the same crystalline lattice and associated by non-ionic and non-covalent bonds. In some embodiments, the cocrystal includes two molecules which are in natural state.

“Cocrystal former” or “crystal former” as used herein is defined as a molecule that forms a cocrystal with Fezolinetant or salts thereof, for example maleic acid, fumaric acid and/or tartaric acid.

As used herein, crystalline Fezolinetant:xinafoic acid is a distinct molecular species. Crystalline Fezolinetant:xinafoic acid may be a co-crystal of Fezolinetant and xinafoic acid. Alternatively, crystalline Fezolinetant:xinafoic acid may be a salt, i.e., Fezolinetant xinafoate. As used herein, “xinafoic acid” is interchangeable with 1-hydroxy-2-naphtoic acid.

As used herein, and unless stated otherwise, the term “anhydrous” in relation to crystalline forms of Fezolinetant, relates to a crystalline form of Fezolinetant which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Fezolinetant of the present disclosure corresponds to a crystalline polymorph of Fezolinetant that is physically separated from the reaction mixture in which it is formed.

As used herein, unless stated otherwise, the XRPD measurements are taken using copper Kα radiation wavelength 1.54187 Å. XRPD peaks reported herein are measured using CuKα radiation, λ=1.54187 Å, typically at a temperature of 25±3° C.

As used herein, unless stated otherwise, ¹³C NMR reported herein are measured at 125 MHz at a magic angle spinning frequency ωᵣ/2π=11 kHz, preferably at a temperature of 293 K±3 K.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

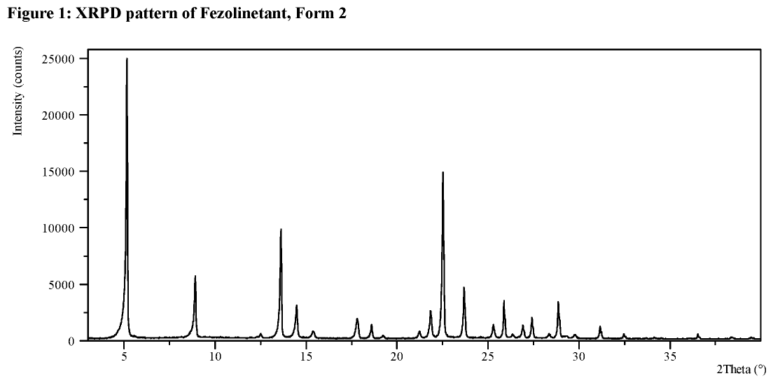

The present disclosure includes a crystalline polymorph of Fezolinetant, designated Form 2. The crystalline Form 2 of Fezolinetant may be characterized by data selected from one or more of the following: an X-ray powder diffraction pattern substantially as depicted in FIG. 1; an X-ray powder diffraction pattern having peaks at 5.1, 8.9, 13.6, 22.5 and 23.7 degrees 2-theta±0.2 degrees 2-theta; and combinations of these data.

Crystalline Form 2 of Fezolinetant may be further characterized by an X-ray powder diffraction pattern having peaks at 5.1, 8.9, 13.6, 22.5 and 23.7 degrees 2-theta±0.2 degrees 2-theta, and also having any one, two, three, four or five additional peaks selected from 14.5, 17.8, 21.2, 21.8 and 25.9 degrees 2-theta±0.2 degrees 2-theta.

Crystalline Form 2 of Fezolinetant may be alternatively characterized by an X-ray powder diffraction pattern having peaks at 5.1, 8.9, 13.6, 14.5, 17.8, 21.2, 21.8, 22.5, 23.7 and 25.9 degrees 2-theta±0.2 degrees 2-theta.

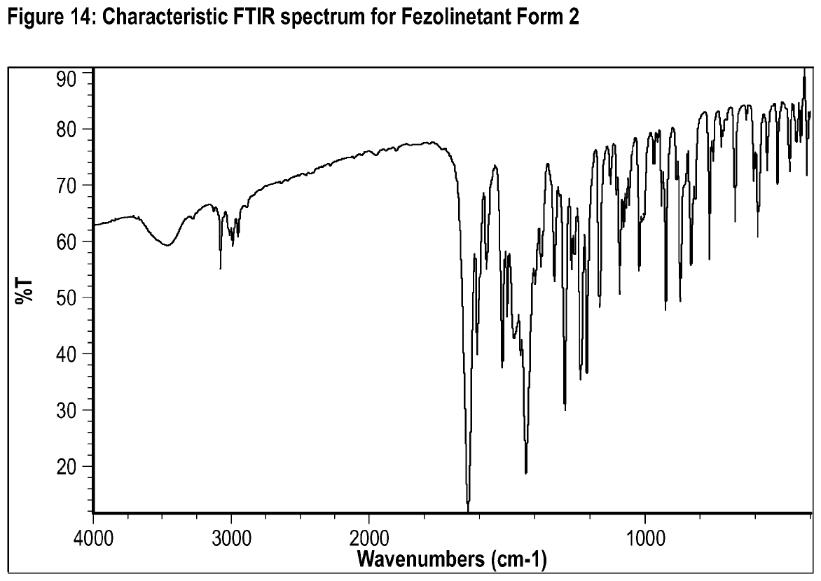

Alternatively, or additionally, according to any embodiment of the present disclosure, Fezolinetant Form 2 can be characterized by FTIR peaks at 1636, 1426, 1285, and 1159±4 cm⁻¹; or by a FTIR spectrum substantially as depicted in FIG. 14.

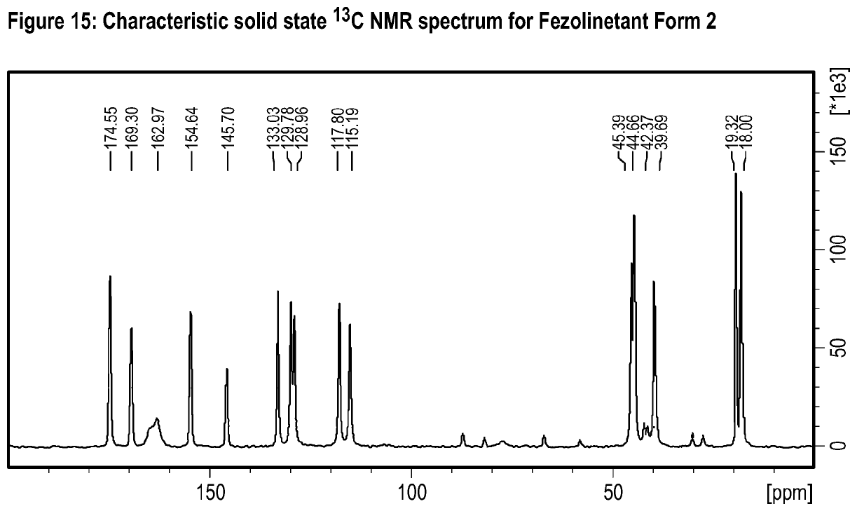

Alternatively, or additionally, according to any embodiment of the present disclosure, Fezolinetant Form 2 can be characterized by a ¹³C solid state NMR spectrum with characteristic peaks: 174.6, 154.6, 133.0, 115.2±0.2 ppm; or by a ¹³C solid state NMR spectrum substantially as depicted in FIG. 15. Alternatively, or additionally, according to any embodiment of the present disclosure, Fezolinetant form 2 can be characterized by a ¹³C solid state NMR spectrum having characteristic chemical shift differences between peaks at 174.6, 154.6, 133.0, 115.2 and a reference peak at 39.7±0.2 ppm of: 134.9, 114.9, 93.3 and 75.5±0.1 ppm.

In one embodiment of the present disclosure, crystalline Form 2 of Fezolinetant is isolated.

Crystalline Form 2 of Fezolinetant may be an anhydrous form.

Crystalline Form 2 of Fezolinetant may be characterized by each of the above characteristics alone or by all possible combinations, e.g., an XRPD pattern having peaks at 5.1, 8.9, 13.6, 22.5 and 23.7 degrees 2-theta±0.2 degrees 2-theta; an XRPD pattern as depicted in, or substantially as depicted in FIG. 1, and combinations thereof.

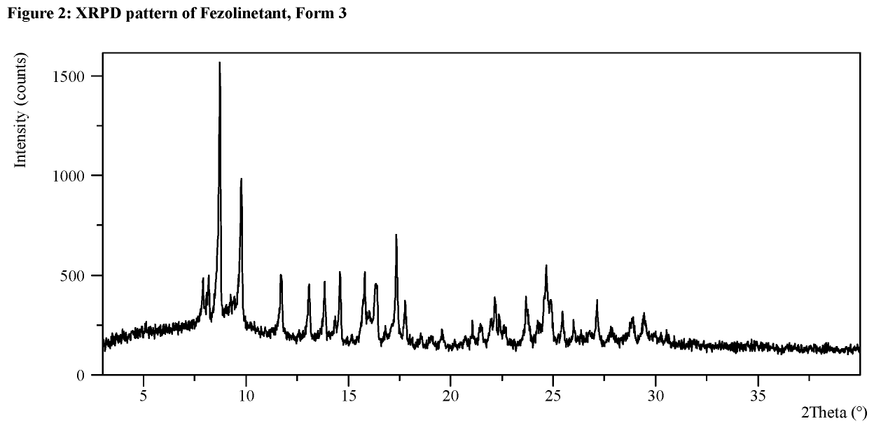

The present disclosure includes a crystalline polymorph of Fezolinetant, designated Form 3. The crystalline Form 3 of Fezolinetant may be characterized by data selected from one or more of the following: an X-ray powder diffraction pattern substantially as depicted in FIG. 2; an X-ray powder diffraction pattern having peaks at 8.7, 9.8, 11.7, 13.1 and 17.4 degrees 2-theta±0.2 degrees 2-theta; and combinations of these data.

Crystalline Form 3 of Fezolinetant may be further characterized by an X-ray powder diffraction pattern having peaks at 8.7, 9.8, 11.7, 13.1 and 17.4 degrees 2-theta±0.2 degrees 2-theta, and also having any one, two, three, four or five additional peaks selected from 7.9, 8.2, 13.8, 17.8 and 23.7 degrees 2-theta±0.2 degrees 2-theta.

Crystalline Form 3 of Fezolinetant may be alternatively characterized by an X-ray powder diffraction pattern having peaks at 7.9, 8.2, 8.7, 9.8, 11.7, 13.1, 13.8, 17.4, 17.8 and 23.7 degrees 2-theta±0.2 degrees 2-theta.

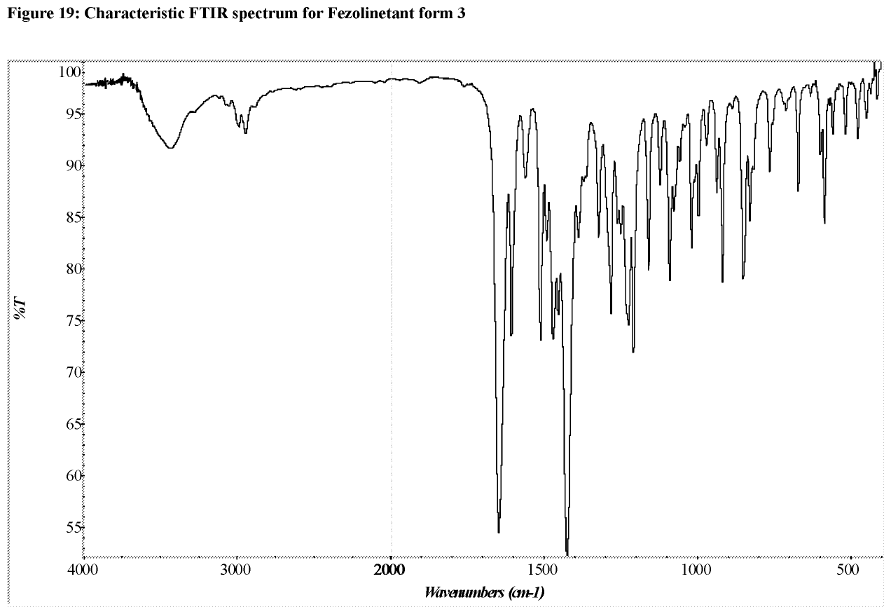

According to any embodiment of the present disclosure, crystalline Form 3 of Fezolinetant may be characterized by an FTIR spectrum having peaks at 1646, 1560, 1423 and 1206±4 cm⁻¹; or an FTIR spectrum substantially as depicted in FIG. 19.

In one embodiment of the present disclosure, crystalline Form 3 of Fezolinetant is isolated.

Crystalline Form 3 of Fezolinetant may be an anhydrous form.

Crystalline Form 3 of Fezolinetant may be characterized by each of the above characteristics alone or by all possible combinations, e.g., an XRPD pattern having peaks at 8.7, 9.8, 11.7, 13.1 and 17.4 degrees 2-theta±0.2 degrees 2-theta; an XRPD pattern as depicted in, or substantially as depicted in, FIG. 2, and combinations thereof

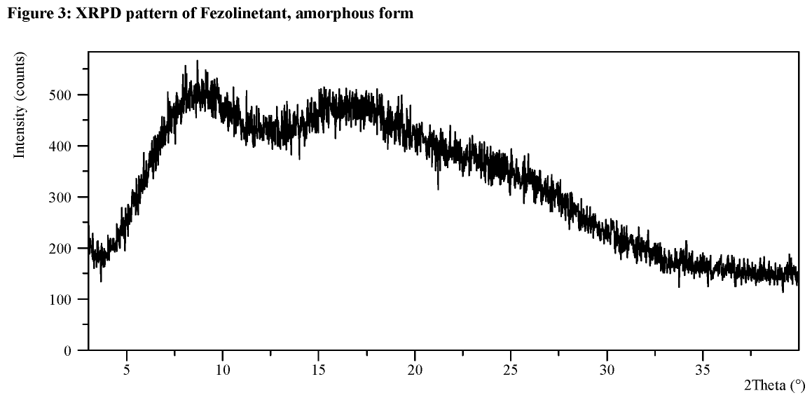

The present disclosure includes an amorphous form of Fezolinetant. The amorphous form of Fezolinetant may be characterized by an X-ray powder diffraction pattern substantially as depicted in FIG. 3.

According to any embodiment of the present disclosure, amorphous Fezolinetant:xinafoic acid may contain: about 0.5 wt % to about 4 wt %, about 1 wt % to about 3 wt %, about 1.5 wt % to about 2.5 wt % of water, or about 2.1 wt % water, preferably as determined by KF (Karl-Fischer titration) analysis.

The present disclosure further includes a crystalline complex of Fezolinetant and xinafoic acid. Crystalline Fezolinetant:xinafoic acid complexes may be a co-crystal of Fezolinetant and xinafoic acid. Alternatively, crystalline Fezolinetant:xinafoic acid may be a salt, i.e., Fezolinetant xinafoate.

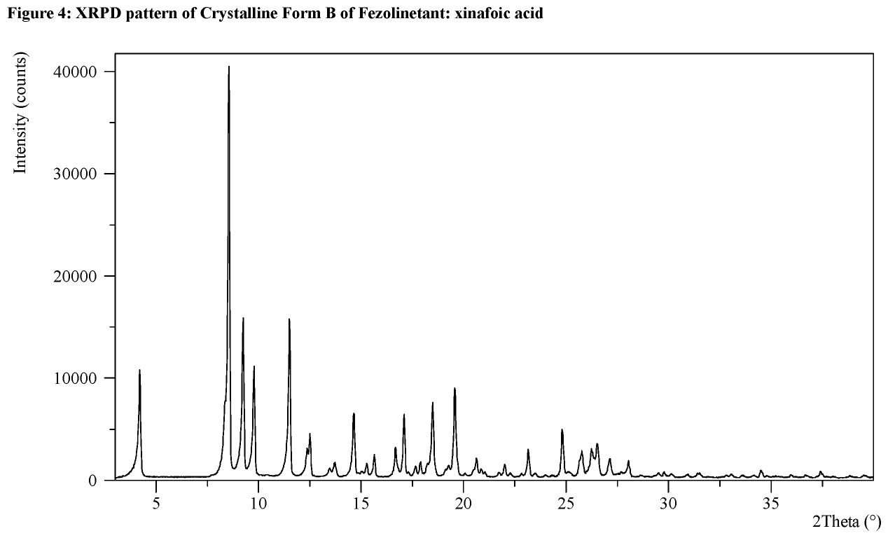

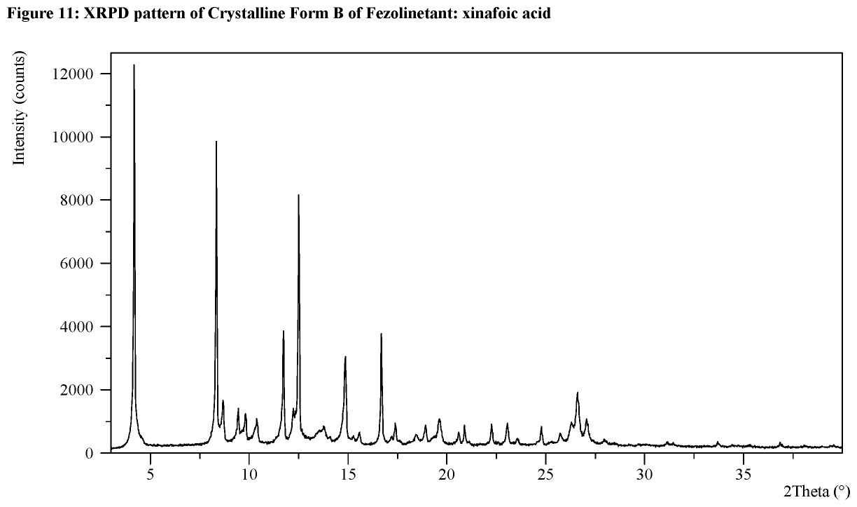

The disclosure further encompasses a crystalline complex of Fezolinetant and xinafoic acid, designated form B. Crystalline Form B of Fezolinetant:xinafoic acid may be characterized by data selected from one or more of the following: an X-ray powder diffraction pattern substantially as depicted in FIG. 4; an X-ray powder diffraction pattern having peaks at 9.2, 9.8, 12.5, 15.7 and 19.6 degrees 2-theta±0.2 degrees 2-theta; and combinations of these data. Alternatively, crystalline Form B of Fezolinetant:xinafoic acid may be characterized by data selected from one or more of the following: an X-ray powder diffraction pattern substantially as depicted in FIG. 11; an X-ray powder diffraction pattern having peaks at 9.2, 9.8, 12.5, 15.7 and 19.6 degrees 2-theta±0.2 degrees 2-theta; and combinations of these data.

Crystalline Form B of Fezolinetant:xinafoic acid complex may be further characterized by an X-ray powder diffraction pattern having peaks at 9.2, 9.8, 12.5, 15.7 and 19.6 degrees 2-theta±0.2 degrees 2-theta, and also having any one, two, three, four or five additional peaks selected from 4.2, 11.5, 13.7, 15.3 and 24.8 degrees 2-theta±0.2 degrees 2-theta.

Crystalline Form B of Fezolinetant:xinafoic acid complex may be alternatively characterized by an X-ray powder diffraction pattern having peaks at 4.2, 9.2, 9.8, 11.5, 12.5, 13.7, 15.3, 15.7, 19.6 and 24.8 degrees 2-theta±0.2 degrees 2-theta

In embodiments of the present disclosure, crystalline Form B of Fezolinetant:xinafoic acid complex is isolated.

In embodiments of the present disclosure, crystalline Form B of Fezolinetant: xinafoic acid complex is a solvate form. Crystalline Form B of Fezolinetant:xinafoic acid complex may be MEK solvate, dichloromethane (DCM) or acetone solvate, particularly a MEK solvate or an acetone solvate. According to any embodiment of the present disclosure, crystalline Form B of Fezolinetant:xinafoic acid complex may be a mono-solvate form, in particular a mono methyl ethyl ketone (MEK) solvate, a mono dichloromethane, or a mono acetone solvate, particularly a mono MEK solvate or a mono acetone solvate

According to any embodiment of the present disclosure, crystalline Fezolinetant:xinafoic acid Form B, optionally in the form of solvate as discussed above, may contain: about 0.05 wt % to about 1.2 wt %, about 0.1 wt % to about 0.5 wt %, about 0.15 wt % to about 0.25% of water, or about 0.2 wt % water, preferably as determined by KF analysis.

According to any embodiment of the present disclosure, crystalline Form B Fezolinetant:xinafoic acid form B may be an acetone solvate, preferably a mono acetone solvate. Fezolinetant:xinafoic acid form B acetone solvate according to any embodiment of the present disclosure may contain: about 6 wt % to about 12 wt % acetone, about 8 wt % to about 10.5 wt % acetone, about 9 wt % to about 9.8 wt % acetone, or about 9.3% of acetone, preferably as determined by GC (Gas chromatography) analysis.

According to any embodiment of the present disclosure, crystalline Form B Fezolinetant:xinafoic acid form B may be a methyl ethyl ketone (MEK) solvate, preferably a mono MEK solvate. Fezolinetant:xinafoic acid form B MEK solvate according to any embodiment of the present disclosure may contain: about 8 wt % to about 15 wt % MEK, about 10 wt % to about 13 wt % MEK, about 11 wt % to about 12 wt % MEK, or about 11.6% MEK, preferably as determined by GC analysis.

According to any embodiment of the present disclosure, crystalline Form B Fezolinetant:xinafoic acid form B may be a dichloromethane (DCM) solvate, preferably a mono DCM solvate. Fezolinetant:xinafoic acid form B solvate according to any embodiment of the present disclosure may contain: about 8 wt % to about 15 wt % DCM, about 10 wt % to about 13 wt % DCM, about 11 wt % to about 12 wt % DCM, or about 13.4% DCM, preferably as determined by GC analysis.

According to an aspect or embodiment of the disclosure, crystalline Form B of Fezolinetant:xinafoic acid complex may be characterized by each of the above characteristics alone or by all possible combinations, e.g., an XRPD pattern having peaks at 9.2, 9.8, 12.5, 15.7 and 19.6 degrees 2-theta±0.2 degrees 2-theta; an XRPD pattern as depicted in, or substantially as depicted in, FIG. 4; and combinations thereof.

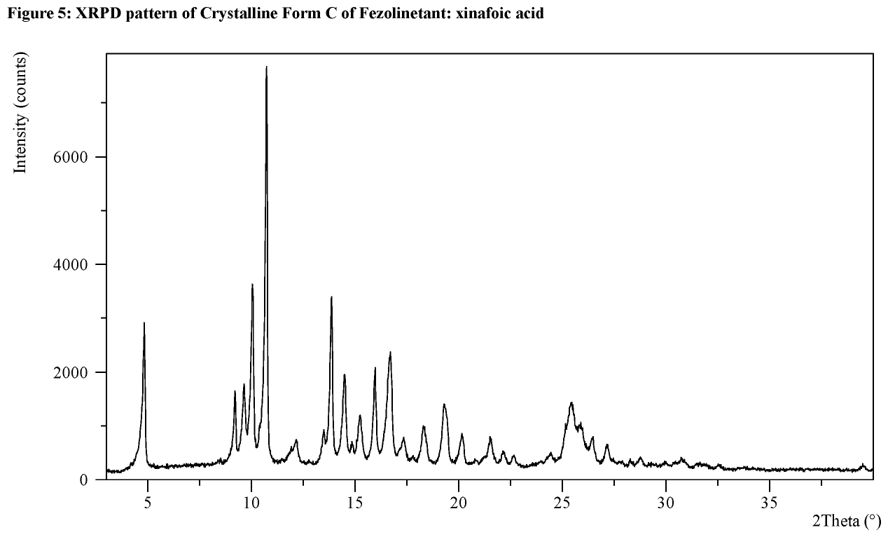

The disclosure further encompasses a crystalline complex of Fezolinetant and xinafoic acid, designated form C. Crystalline Form C of Fezolinetant:xinafoic acid may be characterized by data selected from one or more of the following: an X-ray powder diffraction pattern substantially as depicted in FIG. 5; an X-ray powder diffraction pattern having peaks at 4.8, 10.1, 10.7, 13.9 and 14.5 degrees 2-theta±0.2 degrees 2-theta; and combinations of these data.

Crystalline Form C of Fezolinetant:xinafoic acid complex may be further characterized by an X-ray powder diffraction pattern having peaks at 4.8, 10.1, 10.7, 13.9 and 14.5 degrees 2-theta±0.2 degrees 2-theta, and also having any one, two, three, four or five additional peaks selected from 9.2, 9.6, 15.2, 18.3 and 20.1 degrees 2-theta±0.2 degrees 2-theta.

In any aspect or embodiment of the present disclosure, crystalline Form C of Fezolinetant:xinafoic acid complex may be additionally or alternatively characterized by an X-ray powder diffraction pattern having peaks at 4.8, 9.2, 9.6, 10.1, 10.7, 13.9, 14.5, 15.2, 18.3, and 20.1 degrees 2-theta±0.2 degrees 2-theta.

In embodiments of the present disclosure, crystalline Form C of Fezolinetant:xinafoic acid complex is isolated.

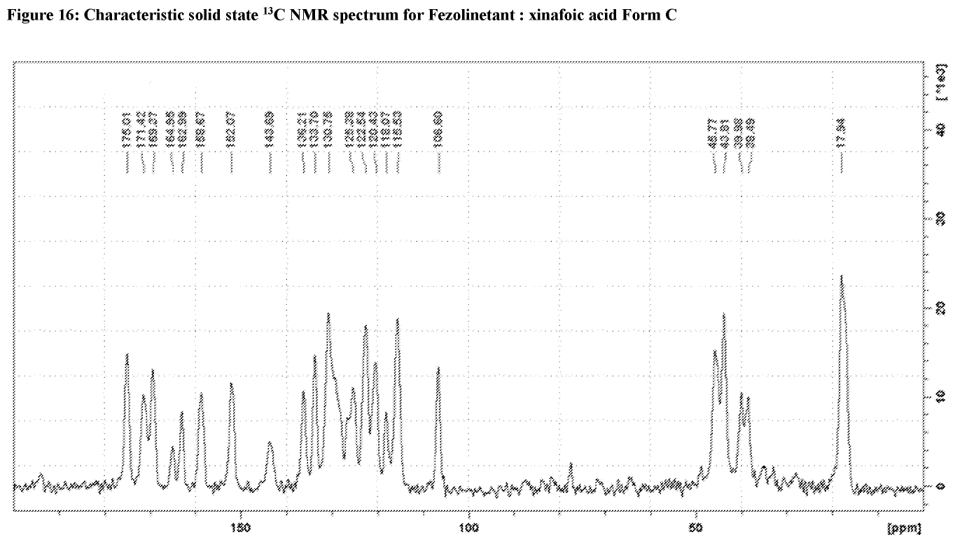

According to any embodiment of the present disclosure, Fezolinetant:xinafoic acid Form C may alternatively or additionally be characterized by a ¹³C solid state NMR spectrum with characteristic peaks at: 175.0, 158.7, 133.7, 106.6±0.2 ppm; or by a ¹³C solid state NMR spectrum substantially as depicted in FIG. 16. According to any embodiment of the present disclosure, Fezolinetant:xinafoic acid Form C may alternatively or additionally be characterized by a ¹³C solid state NMR spectrum having characteristic chemical shift differences between peaks at 175.0, 158.7, 133.7, 106.6 and a reference peak at 38.5±0.2 ppm of: 136.5, 120.2, 95.2 and 68.1±0.1 ppm.

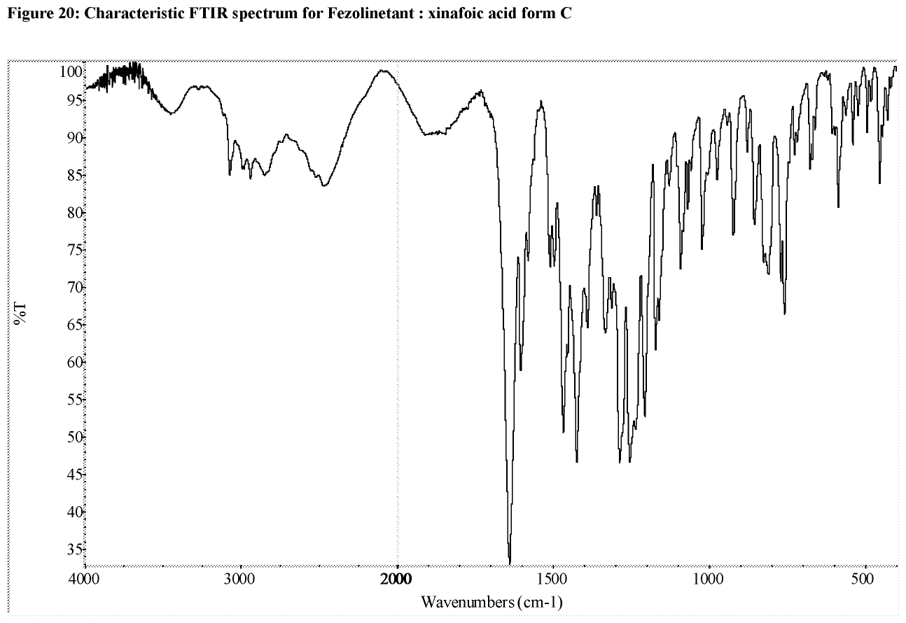

According to any embodiment of the present disclosure, Fezolinetant:xinafoic acid Form C may alternatively or additionally be characterized by an FTIR spectrum having peaks at 1604, 1423, 1204 and 919±4 cm⁻¹; or an FTIR spectrum substantially as depicted in FIG. 20.

In embodiments of the present disclosure, crystalline Form C of Fezolinetant:xinafoic acid complex is an anhydrous form.

Crystalline Form C of Fezolinetant:xinafoic acid complex may be characterized by each of the above characteristics alone or by all possible combinations, e.g., an XRPD pattern having peaks at 4.8, 10.1, 10.7, 13.9 and 14.5 degrees 2-theta±0.2 degrees 2-theta; an XRPD pattern as depicted in, or substantially as depicted in, FIG. 5; and combinations thereof.

The present disclosure includes a crystalline polymorph of Fezolinetant ethanesulfonate (esylate).

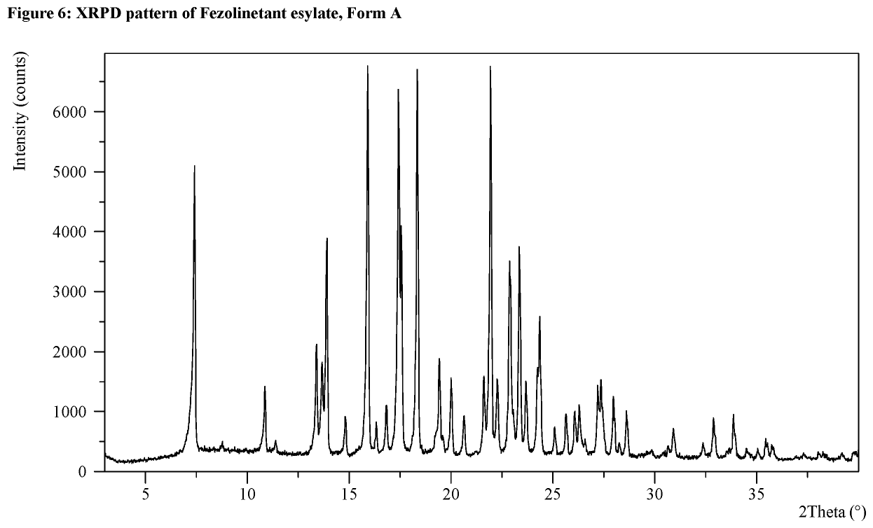

The present disclosure includes a crystalline polymorph of Fezolinetant esylate, designated Form A. The crystalline Form A of Fezolinetant esylate may be characterized by data selected from one or more of the following: an X-ray powder diffraction pattern substantially as depicted in FIG. 6; an X-ray powder diffraction pattern having peaks at 7.4, 15.9, 18.3, 20.0 and 22.5 degrees 2-theta±0.2 degrees 2-theta; and combinations of these data.

Crystalline Form A of Fezolinetant esylate may be further characterized by an X-ray powder diffraction pattern having peaks at 7.4, 15.9, 18.3, 20.0 and 22.5 degrees 2-theta±0.2 degrees 2-theta, and also having any one, two, three, four or five additional peaks selected from 10.9, 13.9, 14.8, 17.4 and 23.4 degrees 2-theta±0.2 degrees 2-theta.

Crystalline Form A of Fezolinetant esylate may be alternatively characterized by an X-ray powder diffraction pattern having peaks at 7.4, 10.9, 13.9, 14.8, 15.9, 17.4, 18.3, 20.0, 22.5 and 23.4 degrees 2-theta±0.2 degrees 2-theta.

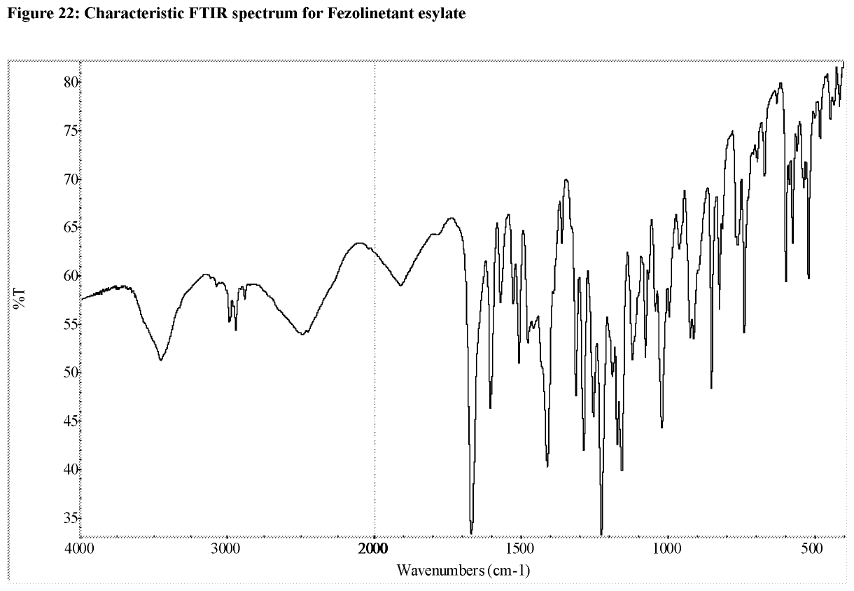

According to any embodiment of the present disclosure, crystalline Form A of Fezolinetant esylate may alternatively or additionally be characterized by an FTIR spectrum having peaks at 1507, 1409, 1285 and 1224±4 cm⁻¹; or an FTIR spectrum substantially as depicted in FIG. 22.

In one embodiment of the present disclosure, crystalline Form A of Fezolinetant esylate is isolated.

Crystalline Form A of Fezolinetant esylate may be an anhydrous form.

Crystalline Form A of Fezolinetant esylate may be characterized by each of the above characteristics alone or by all possible combinations, e.g., an XRPD pattern having peaks at 7.4, 15.9, 18.3, 20.0 and 22.5 degrees 2-theta±0.2 degrees 2-theta; an XRPD pattern as depicted in, or substantially as depicted in, FIG. 6, and combinations thereof

The present disclosure includes a crystalline polymorph of Fezolinetant camphorsulfonate (camsylate).

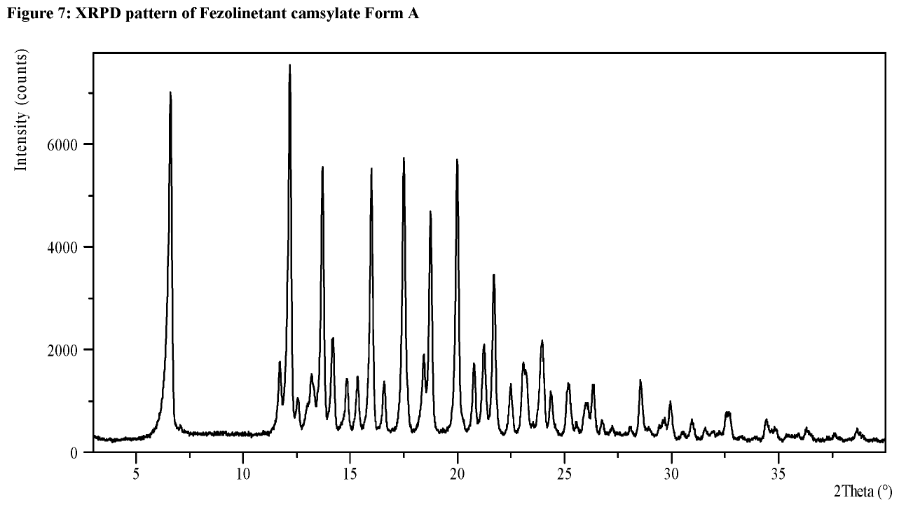

The present disclosure includes a crystalline polymorph of Fezolinetant camsylate, designated Form A. The crystalline Form A of Fezolinetant camsylate may be characterized by data selected from one or more of the following: an X-ray powder diffraction pattern substantially as depicted in FIG. 7; an X-ray powder diffraction pattern having peaks at 6.6, 16.0, 17.5, 20.0 and 22.5 degrees 2-theta±0.2 degrees 2-theta; and combinations of these data.

Crystalline Form A of Fezolinetant camsylate may be further characterized by an X-ray powder diffraction pattern having peaks at 6.6, 16.0, 17.5, 20.0 and 22.5 degrees 2-theta±0.2 degrees 2-theta, and also having any one, two, three, four or five additional peaks selected from 14.9, 15.4, 16.6, 20.8 and 21.7 degrees 2-theta±0.2 degrees 2-theta.

Crystalline Form A of Fezolinetant camsylate may be alternatively characterized by an X-ray powder diffraction pattern having peaks at 6.6, 14.9, 15.4, 16.0, 16.6, 17.5, 20.0, 20.8, 21.7 and 22.5 degrees 2-theta±0.2 degrees 2-theta.

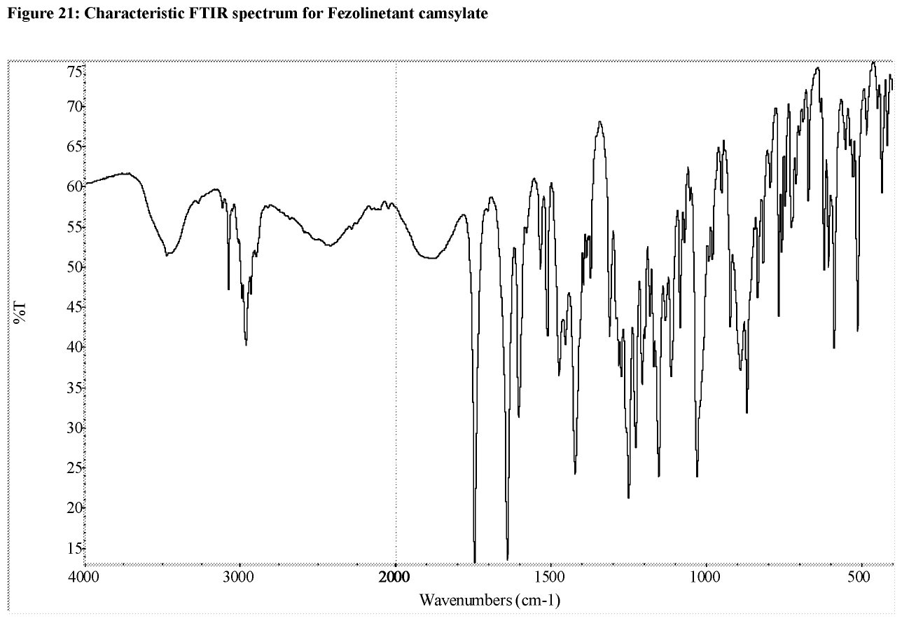

According to any embodiment of the present disclosure, crystalline Form A of Fezolinetant camsylate may alternatively or additionally be characterized by an FTIR spectrum having peaks at 1745, 1511, 1225 and 1081±4 cm⁻¹; or an FTIR spectrum substantially as depicted in FIG. 21.

In one embodiment of the present disclosure, crystalline Form A of Fezolinetant camsylate is isolated.

Crystalline Form A of Fezolinetant camsylate may be an anhydrous form.

Crystalline Form A of Fezolinetant camsylate may be characterized by each of the above characteristics alone or by all possible combinations, e.g., an XRPD pattern having peaks at 6.6, 16.0, 17.5, 20.0 and 22.5 degrees 2-theta±0.2 degrees 2-theta; an XRPD pattern as depicted in, or substantially as depicted in, FIG. 7, and combinations thereof.

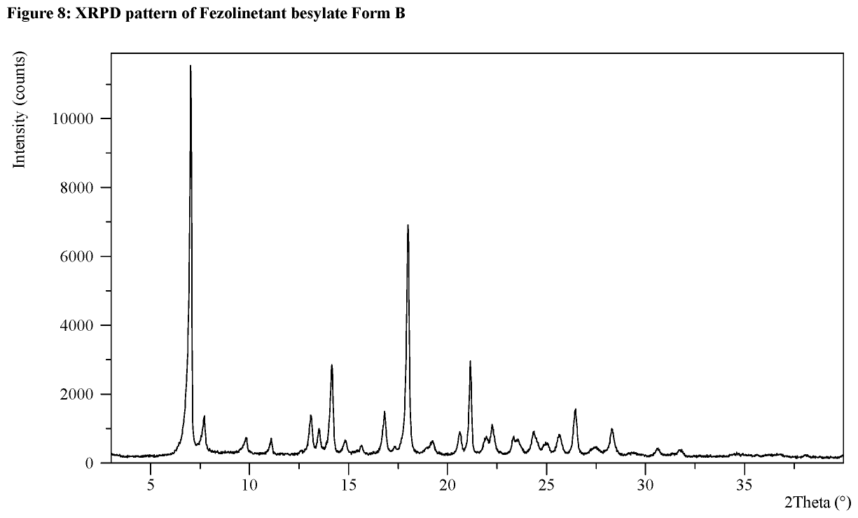

The present disclosure includes a crystalline polymorph of Fezolinetant benzenesulfonate (besylate), designated Form B. According to any aspect or embodiment of the present disclosure, crystalline Form B of Fezolinetant besylate is preferably a salt of Fezolinetant with benzenesulfonic acid. The crystalline Form B of Fezolinetant besylate may be characterized by data selected from one or more of the following: an X-ray powder diffraction pattern substantially as depicted in FIG. 8; an X-ray powder diffraction pattern having peaks at 7.7, 9.8, 11.1, 14.2 and 21.2 degrees 2-theta±0.2 degrees 2-theta; and combinations of these data.

Crystalline Form B of Fezolinetant besylate may be further characterized by an X-ray powder diffraction pattern having peaks at 7.7, 9.8, 11.1, 14.2 and 21.2 degrees 2-theta±0.2 degrees 2-theta, and also having any one, two, three, four or five additional peaks selected from 7.0, 13.1, 13.5, 18.0 and 20.6 degrees 2-theta±0.2 degrees 2-theta.

Crystalline Form B of Fezolinetant besylate may be alternatively characterized by an X-ray powder diffraction pattern having peaks at 7.0, 7.7, 9.8, 11.1, 13.1, 13.5, 14.2, 18.0, 20.6, and 21.2 degrees 2-theta±0.2 degrees 2-theta.

In one embodiment of the present disclosure, crystalline Form B of Fezolinetant besylate is isolated.

According to any embodiment of the disclosure, crystalline Form B of Fezolinetant besylate may contain from about 1.5 wt % to about 4.5 wt % water, about 2 wt % to about 4 wt %, about 2.5 to about 3.8 wt %, or about 3 wt % to about 3.5 wt % or about 3.2 wt % water, preferably as measured by KF analysis.

Crystalline Form B of Fezolinetant besylate may be a hemi-hydrate form.

Crystalline Form B of Fezolinetant besylate may be characterized by each of the above characteristics alone or by all possible combinations, e.g., an XRPD pattern having peaks at 7.7, 9.8, 11.1, 14.2 and 21.2 degrees 2-theta±0.2 degrees 2-theta; an XRPD pattern as depicted in, or substantially as depicted in, FIG. 8, and combinations thereof.

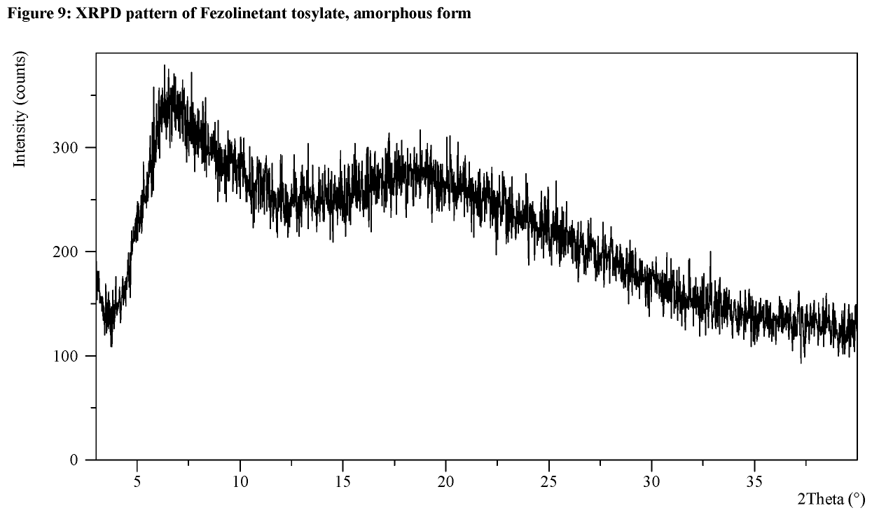

The present disclosure includes an amorphous form of Fezolinetant tosylate. The amorphous form of Fezolinetant tosylate may be characterized by an X-ray powder diffraction pattern substantially as depicted in FIG. 9.

According to any embodiment of the disclosure, amorphous Fezolinetant tosylate may contain from about 0.5 wt % to about 3 wt % water, about 1.0 wt % to about 2.8 wt %, about 1.8 to about 2.5 wt %, or about 2.0 wt % to about 2.2 wt % or about 2.1 wt % water, preferably as measured by KF analysis.

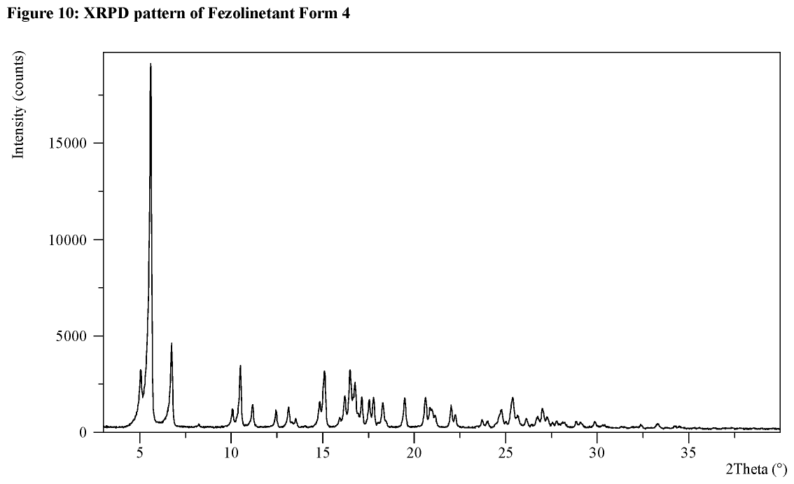

The present disclosure includes a crystalline polymorph of Fezolinetant, designated Form 4. The crystalline Form 4 of Fezolinetant may be characterized by data selected from one or more of the following: an X-ray powder diffraction pattern substantially as depicted in FIG. 10; an X-ray powder diffraction pattern having peaks at 5.6, 6.7, 10.5, 11.2 and 19.5 degrees 2-theta±0.2 degrees 2-theta; and combinations of these data.

Crystalline Form 4 of Fezolinetant may be further characterized by an X-ray powder diffraction pattern having peaks at 5.6, 6.7, 10.5, 11.2 and 19.5 degrees 2-theta±0.2 degrees 2-theta, and also having any one, two, three, four or five additional peaks selected from 12.4, 13.1, 15.1, 16.5 and 17.8 degrees 2-theta±0.2 degrees 2-theta.

Crystalline Form 4 of Fezolinetant may be alternatively characterized by an X-ray powder diffraction pattern having peaks at 5.6, 6.7, 10.5, 11.2, 12.4, 13.1, 15.1, 16.5, 17.8 and 19.5 degrees 2-theta±0.2 degrees 2-theta.

In one embodiment of the present disclosure, crystalline Form 4 of Fezolinetant is isolated.

Crystalline Form 4 of Fezolinetant may be characterized by each of the above characteristics alone or by all possible combinations, e.g., an XRPD pattern having peaks at 5.6, 6.7, 10.5, 11.2 and 19.5 degrees 2-theta±0.2 degrees 2-theta; an XRPD pattern as depicted in, or substantially as depicted in, FIG. 10, and combinations thereof

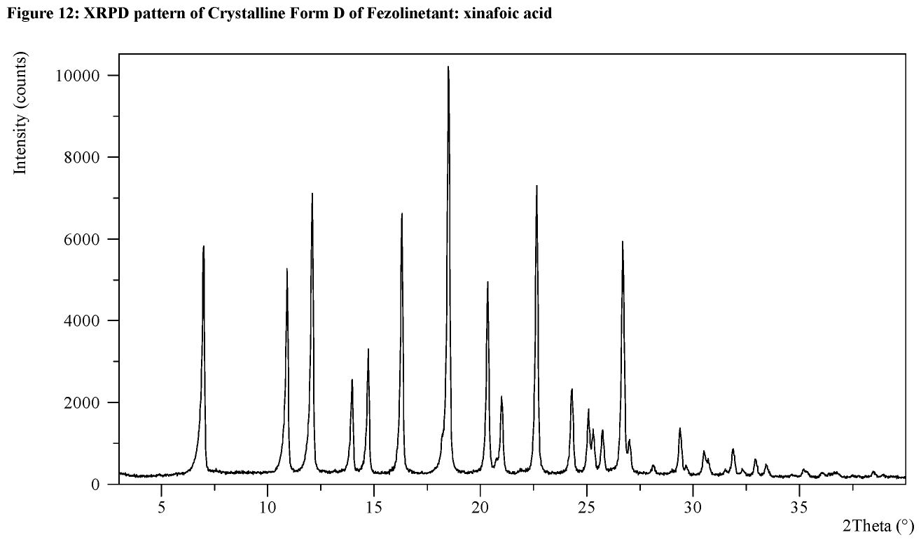

The disclosure further encompasses a crystalline complex of Fezolinetant and xinafoic acid, designated form D. Crystalline Form D of Fezolinetant:xinafoic acid may be characterized by data selected from one or more of the following: an X-ray powder diffraction pattern substantially as depicted in FIG. 12; an X-ray powder diffraction pattern having peaks at 7.0, 12.1, 16.3, 22.6 and 24.3 degrees 2-theta±0.2 degrees 2-theta; and combinations of these data.

Crystalline Form D of Fezolinetant:xinafoic acid complex may be further characterized by an X-ray powder diffraction pattern having peaks at 7.0, 12.1, 16.3, 22.6 and 24.3 degrees 2-theta±0.2 degrees 2-theta, and also having any one, two, three, four or five additional peaks selected from 14.0, 18.5, 21.0, 25.7 and 26.7 degrees 2-theta±0.2 degrees 2-theta.

According to any embodiment of the present disclosure, crystalline Form D of Fezolinetant:xinafoic acid complex may be additionally or alternatively characterized by an X-ray powder diffraction pattern having peaks at 7.0, 12.1, 14.0, 16.3, 18.5, 21.0, 22.6, 24.3, 25.7, and 26.7 degrees 2-theta±0.2 degrees 2-theta.

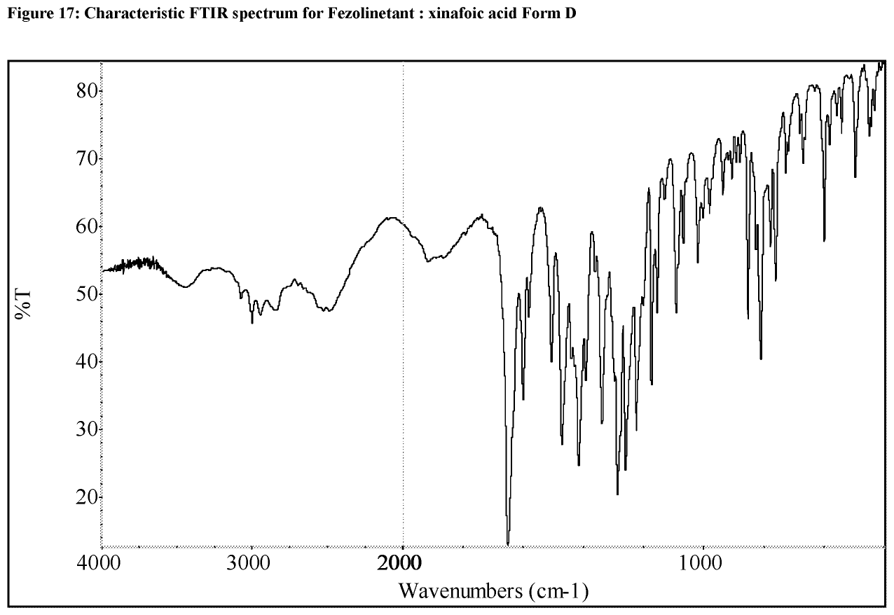

Alternatively, or additionally, according to any embodiment of the present disclosure, Fezolinetant:xinafoic acid Form D can be characterized by FTIR peaks at 1649, 1469, 1336 and 1221±4 cm⁻¹; or by a FTIR spectrum substantially as depicted in FIG. 17.

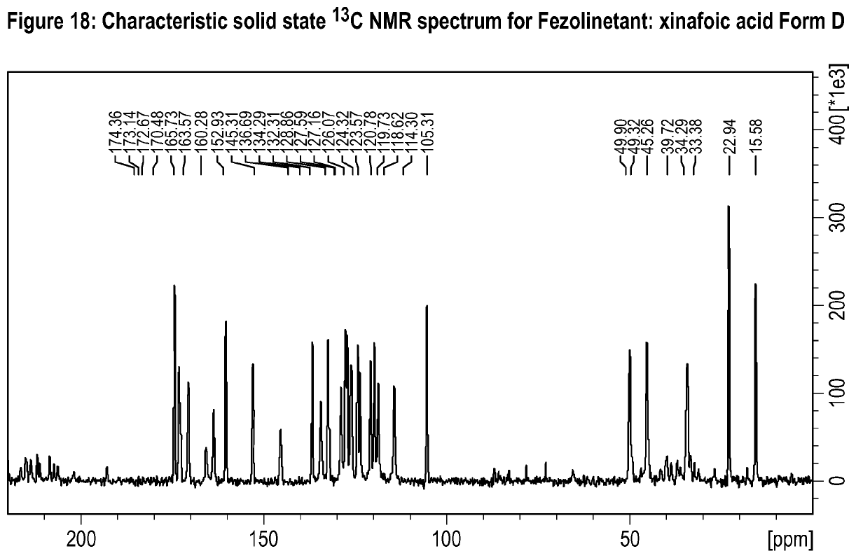

Alternatively, or additionally, according to any embodiment of the present disclosure, Fezolinetant:xinafoic acid Form D can be characterized by a ¹³C solid state NMR spectrum with characteristic peaks: 174.4, 136.7, 49.9, 22.9±0.2 ppm; or by a ¹³C solid state NMR spectrum substantially as depicted in FIG. 18. Alternatively, or additionally, according to any embodiment of the present disclosure, Fezolinetant:xinafoic acid Form D may be characterized by a ¹³C solid state NMR spectrum having characteristic chemical shift differences between peaks at 174.4, 136.7, 49.9, 22.9 and a reference peak at 15.6±0.2 ppm of: 158.8, 121.1, 34.3 and 7.3±0.1 ppm.

In embodiments of the present disclosure, crystalline Form D of Fezolinetant:xinafoic acid complex is isolated.

In embodiments of the present disclosure, crystalline Form D of Fezolinetant:xinafoic acid complex is an anhydrous form.

Crystalline Form D of Fezolinetant:xinafoic acid complex may be characterized by each of the above characteristics alone or by all possible combinations, e.g., an XRPD pattern having peaks at 7.0, 12.1, 16.3, 22.6 and 24.3 degrees 2-theta±0.2 degrees 2-theta; an XRPD pattern as depicted in, or substantially as depicted in, FIG. 12; and combinations thereof.

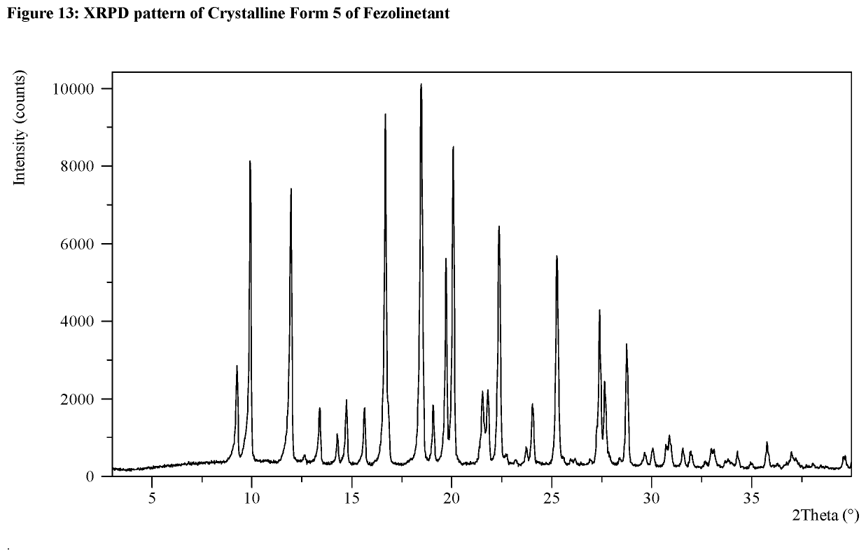

The present disclosure includes a crystalline polymorph of Fezolinetant, designated Form 5. The crystalline Form 5 of Fezolinetant may be characterized by data selected from one or more of the following: an X-ray powder diffraction pattern substantially as depicted in FIG. 13; an X-ray powder diffraction pattern having peaks at 18.5, 20.1, 25.3, 27.4 and 28.7 degrees 2-theta±0.1 degrees 2-theta; and combinations of these data.

Crystalline Form 5 of Fezolinetant may be further characterized by an X-ray powder diffraction pattern having peaks at 18.5, 20.1, 25.3, 27.4 and 28.7 degrees 2-theta±0.1 degrees 2-theta, and also having any one, two, three, four or five additional peaks selected from 13.4, 15.6, 19.1, 22.4 and 30.9 degrees 2-theta±0.1 degrees 2-theta.

Crystalline Form 5 of Fezolinetant may be alternatively characterized by an X-ray powder diffraction pattern having peaks at 13.4, 15.6, 18.5, 19.1, 20.1, 22.4, 25.3, 27.4, 28.7 and 30.9 degrees 2-theta±0.1 degrees 2-theta.

In one embodiment of the present disclosure, crystalline Form 5 of Fezolinetant is isolated.

In embodiments of the present disclosure, crystalline Form 5 of Fezolinetant is an anhydrous form.

Crystalline Form 5 of Fezolinetant may be characterized by each of the above characteristics alone or by all possible combinations, e.g., an XRPD pattern having peaks at 18.5, 20.1, 25.3, 27.4 and 28.7 degrees 2-theta±0.1 degrees 2-theta; an XRPD pattern as depicted in, or substantially as depicted in, FIG. 13, and combinations thereof

In any aspect or embodiment of the present disclosure, any of the solid state forms of Fezolinetant, or cocrystals, or salts of Fezolinetant, e.g. Fezolinetant:xinafoic acid, Fezolinetant esylate, Fezolinetant besylate, and Fezolinetant camsylate, described herein may be polymorphically pure or may be substantially free of any other solid state forms of the subject Fezolinetant, or cocrystals or salts of Fezolinetant, e.g. Fezolinetant:xinafoic acid, Fezolinetant esylate, Fezolinetant besylate, and Fezolinetant camsylate, respectively. In any aspect or embodiment of the present disclosure, any of the solid state forms of Fezolinetant, or cocrystals or salts of Fezolinetant, e.g. Fezolinetant:xinafoic acid, Fezolinetant esylate, Fezolinetant besylate, Fezolinetant camsylate, as described herein may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound, preferably as measured by XRPD. Thus, any of the disclosed crystalline forms of Fezolinetant, or cocrystals or salts of Fezolinetant, e.g. Fezolinetant:xinafoic acid, Fezolinetant esylate, Fezolinetant besylate, Fezolinetant camsylate, described herein may be substantially free of any other solid state forms of the Fezolinetant, or cocrystals or salts of Fezolinetant e.g. Fezolinetant:xinafoic acid, Fezolinetant esylate, Fezolinetant besylate, or Fezolinetant camsylate, respectively, and may contain greater than about 80% (w/w), greater than about 90% (w/w), greater than about 95% (w/w), greater than about 98% (w/w), greater than about 99% (w/w), or about 100% of the subject solid state form of Fezolinetant, or cocrystals or salts of Fezolinetant, e.g. Fezolinetant:xinafoic acid, Fezolinetant esylate, Fezolinetant besylate, or Fezolinetant camsylate.

The above crystalline polymorphs or amorphous form can be used to prepare other crystalline polymorphs of Fezolinetant, Fezolinetant salts and their solid state forms and other cocrystal forms.

The present disclosure encompasses a process for preparing other solid state forms of Fezolinetant, Fezolinetant salts and their solid state forms thereof. The process includes preparing any one of the Fezolinetant (salts) and solid state forms of Fezolinetant by the processes of the present disclosure, and converting it to other Fezolinetant salt(s).

The present disclosure provides the above described crystalline polymorphs and amorphous form of Fezolinetant for use in the preparation of pharmaceutical compositions comprising Fezolinetant and/or crystalline polymorphs thereof. The present disclosure encompasses the use of the above described solid state forms of Fezolinetant and salts thereof, for the preparation of a pharmaceutical composition in the form of a solid dispersion comprising Fezolinetant or salt thereof.

The present disclosure also encompasses the use of crystalline polymorphs or amorphous form of Fezolinetant of the present disclosure for the preparation of pharmaceutical compositions of Fezolinetant and/or crystalline polymorphs thereof. In particular the present disclosure encompasses the above described solid state forms of Fezolinetant and salts thereof, for the preparation of a pharmaceutical composition or formulation, preferably an oral formulation in the form of a solid dispersion comprising Fezolinetant or salt thereof.

The present disclosure includes processes for preparing the above mentioned pharmaceutical compositions. The processes include combining any one or a combination of the crystalline polymorphs and amorphous form of Fezolinetant of the present disclosure with at least one pharmaceutically acceptable excipient.

Pharmaceutical combinations or formulations of the present disclosure contain any one or a combination of the solid state forms of Fezolinetant of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Fezolinetant and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Fezolinetant can be administered. Fezolinetant may be formulated for administration to a mammal, in embodiments to a human, by injection. Fezolinetant can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Fezolinetant and the pharmaceutical compositions and/or formulations of Fezolinetant of the present disclosure can be used as medicaments, in embodiments in the treatment of menopausal hot flashes (HF), and/or other menopausal symptoms such as night sweats, and/or sleep and mood disturbances; polycystic ovary syndrome (PCOS); endometriosis; benign prostate hyperplasia; and uterine fibroids.

The present disclosure also provides methods of treating menopausal hot flashes (HF) and/or other menopausal symptoms such as night sweats, and/or sleep and mood disturbances; polycystic ovary syndrome (PCOS); endometriosis; benign prostate hyperplasia; and uterine fibroids, by administering a therapeutically effective amount of any one or a combination of the crystalline polymorphs of Fezolinetant of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Analytical Methods

### Powder X-ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source =1.54184 Å (Ångström), X'Celerator (2.022° 2θ) detector. Scanning parameters: angle range: 3-40 deg., step size 0.0167, time per step 37 s, continuous scan.

### Differential Scanning Calorimetry (“DSC”)

DSC was performed on instrument Q1000 MDSC TA with a heating rate of 10° C./min and under nitrogen flow of 50 mL/min. Standard aluminum closed pan (with hole) was used, sample mass was 1-5 mg.

Modulated DSC was performed on instrument Q1000 MDSC TA with a heating rate of 2° C./min and modulate temperature amplitude 0.32° C. every 60 sec. Analysis was done under nitrogen flow of 50 mL/min. Standard aluminum closed pan was used, sample mass was 1-5 mg.

### Fourier-Transform Infrared Spectroscopy (“FTIR”)

FTIR spectra were recorded on a Nicolet 6700 interferometer between 4000 cm⁻¹ and 370 cm⁻¹ with resolution of 4 cm⁻¹, in KBr.

### Solid State Nuclear Magnetic Resonance (“ssNMR”)

Solid state NMR analysis was done at Joint Laboratory of Solid State NMR Spectroscopy in Prague, Czech Republic. The spectra were measured at 11.7 T using a Bruker Avance III HD 500 US/WB NMR spectrometer (Karlsruhe, Germany, 2013). The ¹³C CP/MAS NMR spectra employing cross-polarization were acquired using the standard pulse scheme at spinning frequency of 11 kHz. The recycle delay was 8 s and the cross-polarization contact time was 2 ms. The strength of spin-locking fields B1(13C) expressed in frequency units ω½π=γB1 was 64 kHz.

The ¹³C NMR scale was referenced to a-glycine (176.03 ppm). Frictional heating of the spinning samples was offset by active cooling, and the temperature calibration was performed with Pb(NO₃)₂.

The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δv½ was less than 3.5 Hz at 250 ms of acquisition time).

### Karl-Fischer Titration (“KF”)

Water content was determined by coulometric KF titration on Metrohm 831 KF Coulometer.

### Gas Chromatography (“GC”)

GC data is obtained on Agilent 7890A or equivalent instrument with FID detector on a column DB-624, 30 m×0.53 mm, 3μm.

### EXAMPLES

### Preparation of Starting Materials

Fezolinetant can be prepared according to methods known from the literature, for example U.S. Pat. No. 9,422,299.

Fezolinetant besylate can be prepared according to methods known from the literature, for example International Publication No. WO2019/074081.

### Example 1

### Preparation of Fezolinetant Form 2

Fezolinetant (1.0 grams) was weighed into 25 mL round bottomed flask, equipped with condenser and magnetic stirrer. 2-butanol (8 mL) was added and the suspension was heated up to about 70° C. The solution was stirred at 70° C. for 1 hour and cooled down to room temperature (RT). Thick suspension was obtained during cooling at 56° C. Additional quantity of 2-butanol (3 mL) was added and stirred at room temperature (RT) for 16 hours. The precipitate was filtered and analyzed by XRPD. Fezolinetant Form 2 was obtained.

### Example 2

### Preparation of Amorphous Fezolinetant

Fezolinetant (2.0 grams) was dissolved in acetone (80 mL) at room temperature (RT). Solvent was removed by vacuum evaporation (10 mbar) at 50° C. Obtained solid was analyzed by XRPD. Amorphous form of Fezolinetant was obtained.

### Example 3

### Preparation of Fezolinetant Form 3

### Procedure A

Fezolinetant amorphous (0.001 grams) was heated to 100° C. with heating rate 10° C./minute. After it was heated for 5 minutes, solid was analyzed by XRPD. Fezolinetant Form 3 was obtained.

### Procedure B

Fezolinetant amorphous (0.001 grams) was heated to 100° C. with heating rate 10° C./minute under a nitrogen flow in a DSC instrument as described above. After it was heated for 5 minutes, solid was analyzed by XRPD. Fezolinetant Form 3 was obtained.

### Example 4

### Preparation of Fezolinetant Form 3

Fezolinetant amorphous (0.02 grams) suspension in toluene (0.5 mL) was stirred for 2 hours at RT. Obtained solid was filtered and analyzed by XRPD. Fezolinetant Form 3 was obtained.

### Example 5

### Preparation of Fezolinetant Form 3

Fezolinetant amorphous (0.02 grams) suspension in water (0.5 mL) was stirred for 2 hours at RT. Obtained solid was filtered and analyzed by XRPD. Fezolinetant Form 3 was obtained.

### Example 6

### Preparation of Fezolinetant:Xinafoic Acid Complex, Form B

Fezolinetant (1.0 grams) and 1-hydroxy-2-naphthoic acid (1.0 grams) were weighed into 25 mL round bottomed flask, equipped with condenser and magnetic stirrer. Methyl ethyl ketone (MEK; 18 mL) was added and the suspension heated to about 46° C. and stirred for 30 minutes. The obtained solution was cooled to room temperature (RT), resulting in crystallization at about 30° C. The obtained suspension was stirred at RT for 30 minutes. The precipitate was filtered and analyzed by XRPD. Fezolinetant:xinafoic acid complex, Form B was obtained.

### Example 7

### Preparation of Fezolinetant:Xinafoic Acid Complex, Form B

Fezolinetant (0.5 grams) and 1-hydroxy-2-naphthoic acid (0.27 grams) were weighed into 25 mL round bottomed flask, equipped with condenser and magnetic stirrer. Acetone (8 mL) was added and the suspension heated to about 35° C. and stirred for 30 minutes. The obtained solution was cooled to about 10° C., resulting in crystallization at about 23° C. The obtained suspension was stirred at about 10° C. for 30 minutes. The precipitate was filtered and analyzed by XRPD. Fezolinetant:xinafoic acid complex, Form B was obtained.

### Example 8

### Preparation of Fezolinetant:Xinafoic Acid Complex, Form C

Fezolinetant:xinafoic acid complex, Form B (0.5 grams, prepared according to Example 6) was dried at about 100° C. and 10 mbar in a vacuum oven for 2 hours. The product was analyzed by XRPD. Fezolinetant:xinafoic acid complex, Form C was obtained.

### Example 9

### Preparation of Fezolinetant Esylate Form A

Fezolinetant (1.0 grams) was dissolved in ethyl acetate (11 mL) at 60° C. Ethanesulfonic acid (0.28 mL) was added. The obtained solution was cooled to room temperature (RT) and stirred for 3 days. The precipitate was filtered by vacuum and analyzed by XRPD. Fezolinetant esylate Form A was obtained.

### Example 10

### Preparation of Fezolinetant Camsylate Form A

Fezolinetant (0.5 grams) and (1S,4R)-10-camphorsulfonic acid (0.39 grams) were dissolved in ethyl acetate (9 mL) at 60° C. The obtained solution was cooled to RT and methyl tent-butyl ether (9 mL) was added dropwise. The reaction mixture was stirred at RT for 16 hours. The precipitate was filtered by vacuum and analyzed by XRPD. Fezolinetant camsylate Form A was obtained.

### Example 11

### Preparation of Fezolinetant Besylate Form B

Fezolinetant (2.0 grams) was dissolved in ethyl acetate (30 mL) at 60° C. Benzenesulfonic acid monohydrate (1.18 grams) was added to the solution and stirred at 60° C. for 3 hours. The obtained suspension was cooled to room temperature (RT) and stirred for 2 days. The precipitate was filtered and dried at 60° C. for 3 hours.

The obtained solid (0.5 grams) was exposed to 80% relative humidity at RT for 2 days. Solid was analyzed by XRPD. Fezolinetant besylate Form B was obtained.

### Example 12

### Preparation of Fezolinetant:Xinafoic Acid Complex, Form B

Fezolinetant base Form 2 (0.02 grams) and 1-hydroxy-2-naphthoic acid (0.02 grams) were suspended in dichloromethane (6 mL) at 35° C. Solid was filtered and mother liquor was left to evaporate at RT for 2 days. The precipitate was filtered by vacuum and analyzed by XRPD. Fezolinetant:xinafoic acid Form B was obtained.

### Example 13

### Preparation of Amorphous Fezolinetant Tosylate

Fezolinetant (0.5 grams) and p-toluenesulfonic acid monohydrate (0.27 grams) were dissolved in dichloromethane (20 mL) and the solvent evaporated at 50° C. and 10 mbar. Solid residue was analyzed by XRPD. Fezolinetant tosylate amorphous form was obtained.

### Example 14

### Preparation of Fezolinetant Form 4

Amorphous Fezolinetant (0.02 grams) was exposed to vapors of methyl tert-butyl ether (4 mL) at RT for 1 month. Solid was analyzed by XRPD. Fezolinetant Form 4 was obtained.

### Example 15

### Preparation of Fezolinetant:Xinafoic Acid Complex, Form D

Fezolinetant (1.0 grams) and 1-hydroxy-2-naphthoic acid (0.6 grams) were dissolved in methyl isobutyl ketone (9 mL) at 70° C. The solution was stirred at 70° C. for 1 hour and cooled to room temperature (RT), resulting in crystallization at about 40° C. The obtained suspension was stirred at RT for 2 days. The precipitate was filtered and analyzed by XRPD. Fezolinetant:xinafoic acid form D was obtained.

### Example 16

### Preparation of Fezolinetant Form 5

Fezolinetant (1.0 grams) was suspended in water (200 mL) at RT and heated to 90° C. Obtained solution was stirred for 1 hour at 90° C., cooled to room temperature (RT) and stirred for 12 hours. Obtained solid was filtered and analyzed by XRPD. Fezolinetant Form 5 was obtained.

Further aspects and embodiments of the present disclosure are set out in the numbered clauses below:
A1. A crystalline polymorph of Fezolinetant, designated Form 2, which is characterized by data selected from one or more of the following:
   a. an XRPD pattern having peaks at 5.1, 8.9, 13.6, 22.5 and 23.7 degrees 2-theta±0.2 degrees 2-theta;
   b. an XRPD pattern as depicted in, or substantially as depicted in, FIG. 1; and
   c. combinations of these data.
A2. A crystalline polymorph according to Clause A1, designated Form 2, characterized by the XRPD pattern having peaks at 5.1, 8.9, 13.6, 22.5 and 23.7 degrees 2-theta±0.2 degrees 2-theta, and also having one, two, three, four or five additional peaks selected from 14.5, 17.8, 21.2, 21.8 and 25.9 degrees two theta±0.2 degrees two theta.
A3. A crystalline polymorph according to any of Clauses A1 or A2, designated Form 2, wherein the crystalline form is isolated.
A4. A crystalline polymorph according to any of Clauses A1, A2 or A3, designated Form 2, wherein the crystalline form is an anhydrous form.
A5. Crystalline polymorph according to any of Clauses A1, A2, A3 or A4, designated Form 2, which contains: no more than about 20%, no more than about 10%, no more than about 5%, no more than about 2%, no more than about 1% or about 0% of any other crystalline forms of Fezolinetant or Fezolinetant salts.
A6. Crystalline polymorph according to any of Clauses A1, A2, A3, A4, or A5, designated Form 2, which contains: no more than about 20%, no more than about 10%, no more than about 5%, no more than about 2%, no more than about 1% or about 0% of amorphous Fezolinetant or amorphous Fezolinetant salts.
B1. A crystalline polymorph of Fezolinetant, designated Form 3, which is characterized by data selected from one or more of the following:
   a. an XRPD pattern having peaks at 8.7, 9.8, 11.7, 13.1 and 17.4 degrees 2-theta±0.2 degrees 2-theta;
   b. an XRPD pattern as depicted in, or substantially as depicted in, FIG. 2; and
   c. combinations of these data.
B2. A crystalline polymorph according to Clause B1, designated Form 3, characterized by the XRPD pattern having peaks at 8.7, 9.8, 11.7, 13.1 and 17.4 degrees 2-theta±0.2 degrees 2-theta, and also having one, two, three, four or five additional peaks selected from 7.9, 8.2, 13.8, 17.8 and 23.7 degrees two theta±0.2 degrees two theta.
B3. A crystalline polymorph according to any of Clauses B1 or B2, designated Form 3, wherein the crystalline form is isolated.
B4. A crystalline polymorph according to any of Clauses B1, B2 or B3, designated Form 3, wherein the crystalline form is an anhydrous form.
B5. Crystalline polymorph according to any of Clauses B1, B2, B3 or B4, designated Form 3, which contains: no more than about 20%, no more than about 10%, no more than about 5%, no more than about 2%, no more than about 1% or about 0% of any other crystalline forms of Fezolinetant or Fezolinetant salts.
B6. Crystalline polymorph according to any of Clauses B1, B2, B3, B4, or B5, designated Form 3, which contains: no more than about 20%, no more than about 10%, no more than about 5%, no more than about 2%, no more than about 1% or about 0% of amorphous Fezolinetant or amorphous Fezolinetant salts.
C1. A crystalline Fezolinetant:xinafoic acid, designated form B, which is characterized by data selected from one or more of the following:
   a. an XRPD pattern having peaks at 9.2, 9.8, 12.5, 15.7 and 19.6 degrees 2-theta±0.2 degrees 2-theta;
   b. an XRPD pattern as depicted in, or substantially as depicted in, FIG. 4; and
   c. combinations of these data.
C2. A crystalline Fezolinetant:xinafoic acid, designated form B, which is characterized by data selected from one or more of the following:
   a. an XRPD pattern having peaks at 9.2, 9.8, 12.5, 15.7 and 19.6 degrees 2-theta±0.2 degrees 2-theta;
   b. an XRPD pattern as depicted in, or substantially as depicted in, FIG. 11; and
   c. combinations of these data.
C3. Crystalline polymorph according to any of Clauses C1 or C2, designated Form B, characterized by the XRPD pattern having peaks at 9.2, 9.8, 12.5, 15.7 and 19.6 degrees 2-theta±0.2 degrees 2-theta, and also having one, two, three or four additional peaks selected from 4.2, 11.5, 13.7, 15.3 and 24.8 degrees two theta±0.2 degrees two theta. Alternatively, crystalline polymorph according to any of Clauses C1 or C2, designated Form B, characterized by the XRPD pattern having peaks at 9.2, 9.8, 12.5, 15.7 and 19.6 degrees 2-theta±0.2 degrees 2-theta, and also having one, two, three, four or five additional peaks selected from 4.2, 11.5, 13.7, 15.3 and 24.8 degrees two theta±0.2 degrees two theta.
C4. Crystalline polymorph according to any of Clauses C1, C2 or C3, designated Form B, wherein the crystalline form is isolated
C5. Crystalline polymorph according to any of Clauses C1, C2, C3 or C4, designated Form B, wherein the crystalline form is a solvate form.
C6. Crystalline polymorph according to any of Clauses C1, C2, C3, C4 or C5, designated Form B, wherein the crystalline form is MEK solvate.
C7. Crystalline polymorph according to any of Clauses C1, C2, C3, C4 or C5, designated Form B, wherein the crystalline form is acetone solvate.
C8. Crystalline polymorph according to any of Clauses C1, C2, C3, C4, C5, C6 or C7, designated Form B, which is a co-crystal.
C9. Crystalline polymorph according to any of Clauses C1, C2, C3, C4, C5, C6 or C7, designated Form B, which is Fezolinetant xinafoate,
C10. Crystalline polymorph according to any of Clauses C1, C2, C3, C4, C5, C6, C7, C8 or C9, designated Form B, which contains: no more than about 20%, no more than about 10%, no more than about 5%, no more than about 2%, no more than about 1% or about 0% of any other crystalline forms of Fezolinetant or Fezolinetant xinafoate.
C11. Crystalline polymorph according to any of Clauses C1, C2, C3, C4, C5, C6, C7, C8, C9, or C10, designated Form B, which contains no more than about 20%, no more than about 10%, no more than about 5%, no more than about 2%, no more than about 1% or about 0% of Fezolinetant or amorphous Fezolinetant xinafoate.
D1. A crystalline Fezolinetant:xinafoic acid, designated form C, which is characterized by data selected from one or more of the following:
   a. an XRPD pattern having peaks at 4.8, 10.1, 10.7, 13.9 and 14.5 degrees 2-theta±0.2 degrees 2-theta;
   b. an XRPD pattern as depicted in, or substantially as depicted in, FIG. 5; and
   c. combinations of these data.
D2. Crystalline polymorph according to Clause D1, designated Form C, characterized by the XRPD pattern having peaks at 4.8, 10.1, 10.7, 13.9 and 14.5 degrees 2-theta±0.2 degrees 2-theta, and also having one, two, three or four additional peaks selected from 9.2, 9.6, 15.2, 18.3 and 20.1 degrees two theta±0.2 degrees two theta. Alternatively, Crystalline polymorph according to Clause D1, designated Form C, characterized by the XRPD pattern having peaks at 4.8, 10.1, 10.7, 13.9 and 14.5 degrees 2-theta±0.2 degrees 2-theta, and also having one, two, three, four or five additional peaks selected from 9.2, 9.6, 15.2, 18.3 and 20.1 degrees two theta±0.2 degrees two theta.
D3. Crystalline polymorph according to any of Clauses D1 or D2, designated Form C, wherein the crystalline form is isolated.
D4. Crystalline polymorph according to any of Clauses D1, D2 or D3, designated Form C, which is an anhydrous form.
D5. Crystalline polymorph according to any of Clauses D1, D2, D3 or D4, designated Form C, which is a co-crystal.
D6. Crystalline polymorph according to any of Clauses D1, D2, D3 or D4, designated Form C, which is Fezolinetant xinafoate,
D7. Crystalline polymorph according to any of Clauses D1, D2, D3, D4, D5 or D6, designated Form C, which contains: no more than about 20%, no more than about 10%, no more than about 5%, no more than about 2%, no more than about 1% or about 0% of any other crystalline forms of Fezolinetant or Fezolinetant xinafoate.
D8. Crystalline polymorph according to any of Clauses D1, D2, D3, D4, D5, D6, or D7, designated Form C, which contains: no more than about 20%, no more than about 10%, no more than about 5%, no more than about 2%, no more than about 1% or about 0% of Fezolinetant or amorphous Fezolinetant xinafoate.
E1. A crystalline Fezolinetant:xinafoic acid, designated form D, which is characterized by data selected from one or more of the following:
   a. an XRPD pattern having peaks at 7.0, 12.1, 16.3, 22.6 and 24.3 degrees 2-theta±0.2 degrees 2-theta;
   b. an XRPD pattern as depicted in, or substantially as depicted in, FIG. 12; and
   c. combinations of these data.
E2. Crystalline polymorph according to Clause E1, designated Form D, characterized by the XRPD pattern having peaks at 7.0, 12.1, 16.3, 22.6 and 24.3 degrees 2-theta±0.2 degrees 2-theta, and also having one, two, three, four or five additional peaks selected from 14.0, 18.5, 21.0, 25.7 and 26.7 degrees two theta±0.2 degrees two theta.
E3. Crystalline polymorph according to any of Clauses E1 or E2, designated Form D, wherein the crystalline form is isolated.
E4. Crystalline polymorph according to any of Clauses E1, E2 or E3, designated Form D, which is an anhydrous form.
E5. Crystalline polymorph according to any of Clauses E1, E2, E3 or E4, designated Form D, which is a co-crystal.
E6. Crystalline polymorph according to any of Clauses E1, E2, E3 or E4, designated Form D, which is Fezolinetant xinafoate,
E7. Crystalline polymorph according to any of Clauses E1, E2, E3, E4, E5 or E6, designated Form D, which contains: no more than about 20%, no more than about 10%, no more than about 5%, no more than about 2%, no more than about 1% or about 0% of any other crystalline forms of Fezolinetant or Fezolinetant xinafoate.
E8. Crystalline polymorph according to any of Clauses E1, E2, E3, E4, E5 E6, or E7, designated Form D, which contains: no more than about 20%, no more than about 10%, no more than about 5%, no more than about 2%, no more than about 1% or about 0% of amorphous Fezolinetant or amorphous Fezolinetant xinafoate.
F1. A pharmaceutical composition comprising a product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5, E6, E7, or E8, and at least one pharmaceutically acceptable excipient.
F2. Use of a crystalline product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5, E6, E7, or E8,for the preparation of a pharmaceutical composition and/or formulation, preferably wherein the pharmaceutical formulation is a tablet or a capsule.
F3. A process for preparing the pharmaceutical composition according to claim 22, comprising combining a crystalline product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5, E6, E7, or E8,with at least one pharmaceutically acceptable excipient.
F4. A crystalline product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5, E6, E7, or E8, or a pharmaceutical composition according to claim 22, for use as a medicament.
F5. A crystalline product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5, E6, E7, or E8, or a pharmaceutical composition according to Clause F1, for use in the treatment of treatment of menopausal hot flashes (HF) and/or other menopausal symptoms such as night sweats and/or sleep and mood disturbances; polycystic ovary syndrome (PCOS); endometriosis; benign prostate hyperplasia; or uterine fibroids; and preferably for use in the treatment of menopausal hot flashes (HF).
F6. A method of treating menopausal hot flashes (HF) and/or other menopausal symptoms such as night sweats and/or sleep and mood disturbances; polycystic ovary syndrome (PCOS); endometriosis; benign prostate hyperplasia; polycystic ovary syndrome; or uterine fibroids, comprising administering a therapeutically effective amount of a crystalline product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5 E6, E7, or E8, or a pharmaceutical composition according to Clause F1, to a subject in need of the treatment.
F7. Use of a crystalline product according to any of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5 E6, E7, or E8, in the preparation of another solid state form of Fezolinetant or Fezolinetant:xinafoic acid, or Fezolinetant salt.
F8. A process for preparing a solid state form of solid state form of Fezolinetant or Fezolinetant:xinafoic acid, or Fezolinetant salt, comprising preparing any one or a combination of a crystalline product according to any one of Clauses A1, A2, A3, A4, A5, A6, B1, B2, B3, B4, B5, B6, C1, C2, C3, C4, C5, C6, C7, C8, C9, C10, C11, D1, D2, D3, D4, D5, D6, D7, D8, E1, E2, E3, E4, E5, E6, E7, or E8, and converting it to another a solid state form thereof.

1. A crystalline form of Fezolinetant, designated Form 2, which is characterized by data selected from one or more of the following:
(i) an X-ray powder diffraction pattern substantially as depicted in FIG. 1;
(ii) an X-ray powder diffraction pattern having peaks at 5.1, 8.9, 13.6, 22.5 and 23.7 degrees 2-theta±0.2 degrees 2-theta;
(iii) a ¹³C solid state NMR spectrum substantially as depicted in FIG. 15;
(iv) a ¹³C solid state NMR spectrum having characteristic peaks at 174.6, 154.6, 133.0, 115.2±0.2 ppm;
(v) a ¹³C solid state NMR spectrum having characteristic chemical shift differences between peaks at 174.6, 154.6, 133.0, 115.2 and a reference peak at 39.7±0.2 ppm of 134.9, 114.9, 93.3 and 75.5±0.1 ppm; or
(vi) any combination of (i)-(v).

2. A crystalline form of Fezolinetant according to claim 1, which is characterized by an X-ray powder diffraction pattern having peaks at 5.1, 8.9, 13.6, 22.5 and 23.7 degrees 2-theta±0.2 degrees 2-theta, and also having any one, two, three, four or five additional peaks selected from 14.5, 17.8, 21.2, 21.8 and 25.9 degrees 2-theta±0.2 degrees 2-theta, or which is characterized by an X-ray powder diffraction pattern having peaks at 5.1, 8.9, 13.6, 14.5, 17.8, 21.2, 21.8, 22.5, 23.7 and 25.9 degrees 2-theta±0.2 degrees 2-theta.

3. A crystalline form of Fezolinetant according to claim 1, which is further characterized by an FTIR spectrum having peaks at 1636, 1426, 1285, and 1159±4 cm⁻¹; or an FTIR spectrum substantially as depicted in FIG. 14.

4. A crystalline form of Fezolinetant according to claim 1, which is an anhydrous form.

5. A crystalline form of Fezolinetant, designated Form 3, which is characterized by data selected from one or more of the following:
(i) an X-ray powder diffraction pattern substantially as depicted in FIG. 2; or
(ii) an X-ray powder diffraction pattern having peaks at 8.7, 9.8, 11.7, 13.1 and 17.4 degrees 2-theta±0.2 degrees 2-theta.

6. A crystalline form of Fezolinetant according to claim 5, which is characterized by an X-ray powder diffraction pattern having peaks at 8.7, 9.8, 11.7, 13.1 and 17.4 degrees 2-theta±0.2 degrees 2-theta, and also having any one, two, three, four or five additional peaks selected from 7.9, 8.2, 13.8, 17.8 and 23.7 degrees 2-theta±0.2 degrees 2-theta; or which is characterized by an X-ray powder diffraction pattern having peaks at 7.9, 8.2, 8.7, 9.8, 11.7, 13.1, 13.8, 17.4, 17.8 and 23.7 degrees 2-theta±0.2 degrees 2-theta.

7. A crystalline form of Fezolinetant according to claim 5, which is further characterized by an FTIR spectrum having peaks at 1646, 1560, 1423 and 1206±4 cm⁻; or an FTIR spectrum substantially as depicted in FIG. 19.

8. A crystalline form of Fezolinetant according to claim 5, which is an anhydrous form.

9-22. (canceled)

23. A crystalline form of Fezolinetant according to claim 1, which is polymorphically pure.

24. A crystalline form of Fezolinetant according to claim 1, which contains about 20% (w/w) or less of any other crystalline forms of Fezolinetant,or which contains greater than about 80% (w/w) of the subject crystalline form of Fezolinetant.

25. A crystalline form according to claim 1, which contains no more than about 20% (w/w) of amorphous Fezolinetant.

26. A pharmaceutical composition comprising the crystalline form of Fezolinetant according to claim 1, and at least one pharmaceutically acceptable excipient.

27. (canceled)

28. A process for preparing a pharmaceutical composition, comprising combining the crystalline form of Fezolinetant according to claim 1 with at least one pharmaceutically acceptable excipient.

29. A medicament comprising the crystalline form of Fezolinetant according to claim 1.

30. (canceled)

31. A method of treating menopausal hot flashes (HF) and/or other menopausal symptoms such as night sweats and/or sleep and mood disturbances; polycystic ovary syndrome (PCOS); endometriosis; benign prostate hyperplasia; polycystic ovary syndrome; or uterine fibroids, comprising administering a therapeutically effective amount of a crystalline form of Fezolinetant according to claim 1 to a subject in need of the treatment.

32. (canceled)

33. (canceled)
